(19) United States
(12) Patent Application Publication
(10) Pub. No.: US 2011/0257492 A1
(43) Pub. Date: Oct. 20, 2011

(54) METHOD FOR DETERMINING THE STATE OF A SKIN DISORDER USING NEAR INFRARED (NIR) SPECTROSCOPY

(76) Inventors: Tanja Maria Greve, Birkerod (DK); Kristine Birklund Andersen, Stenlose (DK)

(21) Appl. No.: 13/141,559

(22) PCT Filed: Jan. 29, 2010
(86) PCT No.: PCT/DK10/00016

§ 371 Date: Jun. 22, 2011

Related U.S. Application Data
(60) Provisional application No. 61/148,331, filed on Jan. 29, 2009.

(30) Foreign Application Priority Data
Jul. 17, 2009 (DK) PA2009 00882

(51) Int. Cl. A61B 6/00

(52) U.S. Cl. 600/306

(57) ABSTRACT

The invention relates to a non-invasive method for determining the state of a skin disorder using Near Infrared (NIR) spectroscopy as well as a system and an apparatus therefor.

### FIELD OF THE INVENTION

This invention relates to a non-invasive method for determining the state of a skin disorder using Near Infrared (NIR) spectroscopy.

### BACKGROUND OF THE INVENTION

In psoriasis, the increase in keratinocyte proliferation and incomplete differentiation leads to the formation of a highly disordered horny layer and consequently to increased permeability. Up to now the evaluation of the effect of treatment of psoriatic skin has to a large extent been dependent on the subjective evaluation of the appearance of the skin by a physician.

However, biochemical, biophysical and immunopathological studies of psoriatic stratum corneum have demonstrated alterations in lipid metabolism (Moota et al; Interlamellar lipid differences between normal and psoriatic stratum corneum. Acta Venereol Suppl (Stockholm) 1994; 186:131-132).

Raman spectroscopy of biopsies of normal stratum corneum and psoriasic lesions have revealed major spectral differences in the molecular structure of the proteins, whereas psoriatic lesions and normal stratum corneum were found not to differ statistically in their water content (Gniadecka et al., Near-infrared Fourier transform Raman spectroscopic analysis of proteins, water and lipids in intact normal stratum corneum and psoriasis scales, Experimental Dermatology 2004; 13: 391-395). Raman spectroscopy suffers from the disadvantage that the actual measurement must be performed in the dark taking specific precautions such as the need for wearing safety glasses due to the use of a laser beam.

Fourier Transform Near Infrared (FT-NIR) spectroscopy, another non-invasive method, has been used in a study of skin alteration in diabetic patients as compared to control subjects (Geladi et al, A multivariate NIR study of skin alterations in diabetic patients as compared to control subjects, J. Near Infrared Spectrosc. 8; 217-227 (2000)).

A Raman spectrometer measures skin at measurement depths up to several hundred micrometers, which is beyond the boundary of epidermis but within the boundary of dermis, whereas a NIR spectrometer measures within the epidermal layers of the skin, Ph.D. thesis by Tanja Maria Greve, Vibrational spectroscopy as a tool for characterization and investigation of skin, in vivo and in vitro, Department of Chemistry, University of Copenhagen, Spectroscopy and Physical Chemistry, LEO Pharma A/S, August 2008.

There remains a need for a fast, reliable, robust and objective method for determining the state of a skin disorder, which method is non-invasive and comparatively simple to use.

### SUMMARY OF THE INVENTION

The present invention provides a non-invasive method for determining the state of a skin disorder using spectroscopic analysis of the contents and/or structure of one or more of the components water, lipids and proteins in reference skin and in suspect skin, respectively, said method comprising the steps of:
i) subjecting at least one site of reference skin to Near Infrared (NIR) spectroscopy to determine the contents and/or structure of one or more of the components water, lipids and proteins thereof;
ii) subjecting at least one site of test skin to Near Infrared (NIR) spectroscopy to determine the contents and/or structure of one or more of the components water, lipids and proteins thereof;
iii) recording any deviation in the determinations of the contents and/or structure of one or more of the components water, lipids and proteins at the respective skin sites as a measure of the state of the skin disorder.

In another aspect, the present invention relates to a method as outlined in steps (i)-(iii) above for determining the state of a skin disorder using NIR spectroscopic analysis of reference skin and test skin further comprising the step of chemometric analysis of the data obtained.

In a further aspect, the present invention relates to a system for determining the state of a skin disorder using spectroscopic analysis of the contents and/or structure of one or more of the components water, lipids and proteins in reference skin and in test skin, respectively, the system comprising:
i) a Near Infrared (NIR) spectroscopy device suitable for subjecting at least one site of reference skin to Near Infrared (NIR) spectroscopy and suitable for subjecting at least one site of test skin to Near Infrared (NIR) spectroscopy to determine the contents and/or structure of one or more of the components water, lipids and proteins in said sites of reference and test skin, respectively;
ii) a recording device for recording any deviation in the determinations of the contents and/or structure of one or more of the components water, lipids and proteins at the respective skin sites;
iii) an assessment device for assessing a determination of the state of the skin disorder on the basis of said deviation; and
iv) an output device for outputting said determination of the state of the skin disorder.

In a further aspect the present invention relates to an apparatus for determining the state of a skin disorder, the apparatus comprising:
i) an input device for interfacing with a Near Infrared (NIR) spectroscopy device for receiving determinations of the contents and/or structure of one or more of the components water, lipids and proteins in at least one site of reference and test skin, respectively, as obtained by NIR spectroscopy applied to said skin sites by the NIR spectroscopy device;
ii) a recording device for recording any deviation in the determinations of the contents and/or structure of one or more of the components water, lipids and proteins at the respective skin sites;
iii) an assessment device for assessing a determination of the state of the skin disorder on the basis of said deviation;
iv) an output device for outputting said determination of the state of the skin disorder.

### BRIEF DESCRIPTION OF THE DRAWINGS

The invention is disclosed in further detail with reference to the drawings, in which

FIG. 1 shows a FT-NIR spectrum of healthy skin in vivo;

FIG. 2 shows a FT-NIR spectrum of an untreated psoriasis transplant on a SCID mouse;

FIG. 3 shows a scoreplot of PC1 versus PC2 from a PCA of FT-NIR spectra from betamethasone treated SCID mice and untreated healthy skin in vivo.

FIG. 4 shows the scoreplot of PC1 versus PC2 from a PCA of FT-NIR spectra from placebo treated SCID mice and untreated healthy skin in vivo.

FIG. 5 shows the FT-NIR spectra of asymptomatic and lesional skin from 7 psoriasis patients (2 patients with PASI score 4, 1 patient with a PASI score 5, 1 patient with PASI score 6, 1 patient with PASI score 7, 1 patient with PASI score 8 and 1 patient with PASI score 10).

FIG. 6 shows the PLS correlation between measured and predicted PASI scores based on the FT-NIR spectra of asymptomatic and lesional skin from 7 psoriasis patients.

### DETAILED DESCRIPTION OF THE INVENTION

NIR spectroscopy is a fast, reliable and robust method for obtaining information of the contents of essential elements of the skin. NIR spectroscopy measures at skin depths of about 150-200 micrometers, i.e. within the epidermis. Thus it has surprisingly been found that the contents and/or structure of the bands, measured as band width, band intensity, band shape and/or band position, of one or more of the components water, lipids, and proteins obtained by NIR spectroscopy is indicative of the condition of the skin. In particular the water bands and/or lipid bands obtained by NIR are strong and/or indicative of the condition of the skin. It has been shown that spectroscopic information on the contents and/or structure of especially water and/or lipids is a suitable marker for the condition of the skin, and more particularly the condition of psoriatic skin. As described in detail in examples 1 and 2 below, the NIR spectra of psoriasis transplants and lesional skin on a psoriasis patient show less water and an altered lipid structure as compared to the spectra of healthy reference skin.

### DEFINITIONS

In the present context the term “NIR” refers to Near Infrared spectroscopy, which method is based on measurements of the sample absorption of the incident Near Infrared radiation.

In the present context the term “skin disorder” refers to any condition of the skin different from healthy reference skin. In particular, the term refers to a skin disease such as psoriasis, atopic dermatitis, contact dermatitis, seborrhoic dermatitis, urticaria, rosacea, systemic lupus erythematosus, skin atrophy, skin ageing, pruritis or eczema.

The term “state of a skin disorder” refers to the condition or severity of a skin disorder at any given point in time.

In the present context the term “reference skin” refers to healthy skin or to skin afflicted by a skin disorder of different severity than suspect skin.

In the present context the term “severity” refers to a judgement based on the PASI score (in case of psoriasis), which sums up the assessments for erythema, thickness & desquamation of a given skin area, or based on similar evaluation scores applicable to other skin diseases.

The term “PASI score” refers to a system of visually assessing the severity and symptoms of psoriasis used routinely by dermatologists. The PASI (Psoriasis Area and Severity Index) score is calculated according to the following formulae:

Arms 0.2(R+T+S)E=X

Trunk 0.3(R+T+S)E=Y

Legs 0.4(R+T+S)E=Z

wherein R=score for erythema, T=score for thickness, S=score for desquamation and E=score for extent. The score is assessed on a scale from 0 to 4 as follows: 0=no symptoms, 1=<10%, 2=10-29%, 3=30-49% and 4=50-69% involvement. The sum of X+Y+Z gives the total PASI score.

PASI is also used to assess the efficacy of a given treatment (Langley et al, Evaluating Psoriasis Area and Severity Index, Psoriasis Global Assesment, and Lattice System Physicians Global Assesment, J Am Acad Dermatol 51, 563-569 (2004)). “Local PASI score” refers to a PASI score where the area is not taken into account. That is to say that the three parameters erythema, thickness & desquamation are used to determine the local score and each parameter is assigned a score from 0 to 4 so that the total score is on a scale from 0 to 12 (0 being not afflicted with psoriasis and 12 being severely afflicted with psoriasis).

In the present context the term “healthy skin” refers to any skin not afflicted with any kind of disorder or disease. “Healthy skin” may originate from a person not afflicted by any skin disorder or may originate from an asymptomatic part of the skin of a person afflicted by a skin disorder.

In the present context the term “test skin” refers to any skin known or suspected to be afflicted with a skin disorder and subjected to testing by the present spectroscopic method.

In the present context the term “negative reference” or “negative control” refers to a reference known from previous experience to give a negative result. A negative control indicates a result obtained when a test does not produce a measurable change compared to a previously established reference baseline. The negative reference is typically healthy skin as defined above.

In the present context the term “positive reference” or “positive control” refers to a reference known from previous experience to give a positive result. A positive control confirms that the basic conditions of the experiment were able to produce a measurable change compared to a baseline result. The positive reference is typically skin afflicted with a skin disorder.

In the present context the term “contents” of water, lipids and/or proteins refers to the amount of water, lipids and/or proteins, respectively, present in the skin of interest as determined by the NIR spectrometer.

In the present context the term “structure of water” refers to whether said water is present in free form or bound to or associated with e.g. the lipids or proteins of skin.

In the present context the term “structure of lipids” refers to the composition as well as the spatial arrangement of the lipids of interest.

In the present context the term “structure of proteins” refers to the composition as well as the spatial arrangement—i.e. the secondary structure—of the proteins of interest.

In the present context the term “determination of the contents and/or structure” refers to any value established in the form of at least one of the following: band width, band intensity, band shape and/or band position of the spectra obtained.

In the present context the term “deviation” refers to any difference in value of the contents and/or structure of any one or more of the components water, lipids, and proteins obtained by the method according to the invention between the contents and/or structure in reference skin and the contents and/or structure in test skin, measured as a deviation in at least one of the following: band width, band intensity, band shape and/or band position, which deviation is significant.

The term “significant” refers in the present context to a difference or deviation of 5% or more, such as 10% or more, 15% or more, such as 20% or more, 25% or more, such as 30% or more, 35% or more, such as 40% or more, 45% or more, such as 50% or more.

In the present context the term “PCA” stands for “Principal Component Analysis” (K. H. Esbensen, Multivariate Data Analysis, 5^{th} edition, Camo Process, Norway, 2002). Hence “PCA” refers to a tool in exploratory data analysis used to reduce multidimensional data sets to lower dimensions for analysis such that the greatest variation of the dataset is explained by the first principal component (or PC1), the second greatest variation is explained by the second principal component (PC2), and so on. The principal components are linear combinations of the original variables in the dataset. The data analysis may be conducted using the Unscrambler software (CAMO, Norway).

In the present context, the term “PLS” stands for Partial Least Squares (L. Staahle et al., Multivariate data analysis and experimental design in biomedical research, in Progress in Medicial Chemistry vol 25, G. P. Ellis et al (Eds.), Elsevier Science Publishers, 1998, pp. 291-338. Like PCA, PLS refers to a tool in exploratory data analysis used to disclose structure in the data set in view of external information. Hence, PLS can be used to find a potential correlation between spectral deviations and for instance a given PASI score. The data analysis may be conducted using the Unscrambler software (CAMO, Norway).

### EMBODIMENTS

In one embodiment of the invention the reference skin refers to healthy skin or to skin afflicted by a skin disorder of less severity than test skin in which case the reference skin is used as a negative reference.

In another embodiment of the invention the reference skin refers to skin afflicted by a skin disorder of greater severity than test skin in which case the reference skin is used as a positive reference.

In an embodiment of the invention said reference skin is healthy skin.

In an embodiment the method according to the invention is for determining the state of a skin disorder using spectroscopic analysis of the contents and/or structure of water in reference skin and in test skin, respectively, said method comprising the steps of:
i) subjecting at least one site of reference skin to Near Infrared (NIR) spectroscopy to determine the contents and/or structure of water thereof;
ii) subjecting at least one site of test skin to Near Infrared (NIR) spectroscopy to determine the contents and/or structure of water thereof;
iii) recording any deviation in the determinations of the contents and/or structure of water at the respective skin sites as a measure of the state of the skin disorder.

The water bands obtained by NIR spectroscopy are distinct and of significant intensity. Thus the water bands obtained by NIR spectroscopy have shown to readily display the condition of the skin. Thus any deviation in the water bands in the form of a deviation in band width, band intensity, band shape and/or band position may provide information about alterations in water structure and changes in water content which are expected to be important in the determination of skin states. The shape and relative intensities of the water bands are indicative of the binding of the water as well as of the concentration thereof.

In an embodiment the method according to the invention is for determining the state of a skin disorder using spectroscopic analysis of the contents and/or structure of lipids in reference skin and in test skin, respectively, said method comprising the steps of:
i. subjecting at least one site of reference skin to Near Infrared (NIR) spectroscopy to determine the contents and/or structure of lipids thereof;
ii. subjecting at least one site of test skin to Near Infrared (NIR) spectroscopy to determine the contents and/or structure of lipids thereof;
iii. recording any deviation in the determinations of the contents and/or structure of lipids at the respective skin sites as a measure of the state of the skin disorder.

It has been found that the lipid bands are important in the determination of the state of skin. Thus any deviation in the lipid bands in the form of a deviation in band width, band intensity, band shape and/or band position may provide information about alterations in lipid structure and changes in lipid content which are expected to be important in the determination of skin states. The shape and relative intensities of the lipid bands are indicative of the spatial arrangement of the lipids as well as of the concentration thereof.

In an embodiment the method according to the invention is for determining the state of a skin disorder using spectroscopic analysis of the contents and/or structure of water and lipids in reference skin and in test skin, respectively, said method comprising the steps of:
i) subjecting at least one site of reference skin to Near Infrared (NIR) spectroscopy to determine the contents and/or structure of water and lipids, respectively, thereof;
ii) subjecting at least one site of test skin to Near Infrared (NIR) spectroscopy to determine the contents and/or structure of water and lipids, respectively, thereof;
iii) recording any deviation in the determinations of the contents and/or structure of water and lipids at the respective skin sites as a measure of the state of the skin disorder.

Using chemometric analysis e.g. as described in Greve et al (Greve et al, ATR-FTIR, FT-NIR and near-FT-Raman spectroscopic studies of molecular composition in human skin in vivo and pig ear skin in vitro, Spectroscopy 22, 437-457 (2008)) initial test studies have shown that both water and lipid bands are important in the separation of NIR spectra of transplants of diseased skin and NIR spectra of reference skin. Thus the contents and/or structure of water and lipids are expected to be important in the determination of the state of skin.

In an embodiment the method according to the invention is for determining the state of a skin disorder using spectroscopic analysis of the contents and/or structure of proteins in reference skin and in test skin, respectively, said method comprising the steps of:
i) subjecting at least one site of reference skin to Near Infrared (NIR) spectroscopy to determine the contents and/or structure of proteins thereof;
ii) subjecting at least one site of test skin to Near Infrared (NIR) spectroscopy to determine the contents and/or structure of proteins thereof;
iii) recording any deviation in the determinations of the contents and/or structure of proteins at the respective skin sites as a measure of the state of the skin disorder.

In an embodiment the method according to the invention is for determining the state of a skin disorder using spectroscopic analysis of the contents and/or structure of water and proteins in reference skin and in test skin, respectively, said method comprising the steps of:
i) subjecting at least one site of reference skin to Near Infrared (NIR) spectroscopy to determine the contents and/or structure of water and proteins, respectively, thereof;
ii) subjecting at least one site of test skin to Near Infrared (NIR) spectroscopy to determine the contents and/or structure of water and proteins, respectively, thereof;
iii) recording any deviation in the determinations of the contents and/or structure of water and proteins at the respective skin sites as a measure of the state of the skin disorder.

In an embodiment the method according to the invention is for determining the state of a skin disorder using spectroscopic analysis of the contents and/or structure of lipid and proteins in reference skin and in test skin, respectively, said method comprising the steps of:
i) subjecting at least one site of reference skin to Near Infrared (NIR) spectroscopy to determine the contents and/or structure of lipids and proteins, respectively, thereof;
ii) subjecting at least one site of test skin to Near Infrared (NIR) spectroscopy to determine the contents and/or structure of lipids and proteins, respectively, thereof;
iii) recording any deviation in the determinations of the contents and/or structure of lipids and proteins at the respective skin sites as a measure of the state of the skin disorder.

In an embodiment the method according to the invention is for determining the state of a skin disorder using spectroscopic analysis of the contents and/or structure of water, lipids and proteins in reference skin and in test skin, respectively, said method comprising the steps of:
i) subjecting at least one site of reference skin to Near Infrared (NIR) spectroscopy to determine the contents and/or structure of water, lipids and proteins, respectively, thereof;
ii) subjecting at least one site of test skin to Near Infrared (NIR) spectroscopy to determine the contents and/or structure of water, lipids and proteins, respectively, thereof;
iii) recording any deviation in the determinations of the contents and/or structure of water, lipids and proteins at the respective skin sites as a measure of the condition of the skin disorder.

In an embodiment the method according to the invention is for determining the state of psoriasis, contact dermatitis, rosacea, systemic lupus erythematosus, urticaria, atrophied, eczematous or atopic dermatitis skin. Thus it has been shown that the method according to the invention allows a fast, reliable, robust and objective method for determining the state of a skin disorder, such as the above mentioned skin disorders.

In an embodiment the method according to the invention is for determining the state of psoriasis skin.

In an embodiment the method according to the invention is for determining the effect of treatment of skin disorders. In this embodiment the method comprises repeating steps (i)-(iii) of any one of the methods indicated above at least once at a later point in time and determining any change in the state of the skin disorder being treated by comparing the determinations recorded at said later point in time with the original determinations.

The method according to the invention is non-invasive and comparatively simple to use and consequently presents a major advance in the evaluation of a skin disorder. Up till now the evaluation of the effect of treatment of skin disorders has to a large extent been dependent of the subjective evaluation by the physician of the appearance of the patient's skin (such as by determining the PASI score) which may result in a less than optimal treatment regime. By the use of the method according to the invention an objective evaluation has become possible.

In an embodiment the method according to the invention is for determining the effect of treatment of psoriasis, contact dermatitis, rosacea, systemic lupus erythematosus, urticaria, atrophied, eczematous or atopic dermatitis skin.

In an embodiment the method according to the invention is for determining the effect of treatment of psoriasis skin.

In an embodiment the method according to the invention is for determining the state of a skin disorder using spectroscopic analysis of reference skin and test skin further comprising the step of chemometric analysis of the data obtained.

Thus by using e.g. Principal Component Analysis of the data sets obtained from NIR spectroscopy of reference skin and of test skin, respectively, a chemometric analysis of the data sets obtained may provide a fast overview of any deviation between the NIR spectra of reference skin and of test skin as well as the effect of any treatment of test skin. If initially a significant difference in the spectra of reference skin and of test skin exists, and said difference is reduced by treatment of test skin, this finding is indicative of an effect of said treatment.

Chemometric analyses have shown that one or more of the water, lipid and protein bands are important in the separation of NIR spectra of diseased skin and of reference skin. In particular it has been shown that chemometric analysis of water and lipid bands of NIR spectra of psoriasis transplants and NIR spectra of reference skin are suitable for determining the state of psoriasis.

In an embodiment of the invention said chemometric analysis may be used for determining the state of psoriasis skin.

In an embodiment of the invention said chemometric analysis may be used for determining the effect of treatment of psoriasis skin.

In an embodiment of the invention the NIR spectroscopy is Fourier Transform Near Infrared (FT-NIR) spectroscopy. The FT based measurement procedure has a number of advantages, most notably a markedly increased sensitivity and wavelength stability compared to the dispersive-type instruments. Also the FT spectrometer is fast and a spectrum can be obtained within a few seconds.

Another aspect of the invention is a system for determining the state of a skin disorder using spectroscopic analysis of the contents and/or structure of one or more of the components water, lipids and proteins in reference skin and in test skin, respectively, the system comprising:
i) a Near Infrared (NIR) spectroscopy device suitable for subjecting at least one site of reference skin to Near Infrared (NIR) spectroscopy and suitable for subjecting at least one site of test skin to Near Infrared (NIR) spectroscopy to determine the contents and/or structure of one or more of the components water, lipids and proteins in said sites of reference and suspect skin, respectively;
ii) a recording device for recording any deviation in the determinations of the contents and/or structure of one or more of the components water, lipids and proteins at the respective skin sites;
iii) an assessment device for assessing a determination of the state of the skin disorder on the basis of said deviation; and
iv) an output device for outputting said determination of the state of the skin disorder.

In an embodiment thereof said system further comprises a user interface for receiving user input concerning the treatment of the test skin site, the output device further being adapted to monitor the effect of said treatment.

Another aspect of the invention is an apparatus for determining the state of a skin disorder, the apparatus comprising:
i) an input device for interfacing with a Near Infrared (NIR) spectroscopy device for receiving determinations of the contents and/or structure of one or more of the components water, lipids and proteins in at least one site of reference and test skin, respectively, as obtained by NIR spectroscopy applied to said skin sites by the NIR spectroscopy device;
ii) a recording device for recording any deviation in the determinations of the contents and/or structure of one or more of the components water, lipids and proteins at the respective skin sites;
iii) an assessment device for assessing a determination of the state of the skin disorder on the basis of said deviation;
iv) an output device for outputting said determination of the state of the skin disorder.

In an embodiment thereof said apparatus further comprises a user interface for receiving user input concerning the treatment of the test skin site, the output device being further adapted to monitor the effect of said treatment.

### EXAMPLES

### Example 1

A Bruker Equinox 55 spectrometer equipped with a DLATGS detector (available from Bruker) was used to obtain FT-NIR spectra in the region 12500-4000 cm⁻¹. The spectrometer was coupled with a fiber-optic probe which has a circular measurement area of approximately 4 mm in diameter. The measurements are based on diffuse reflectance. For each healthy human skin sample in vivo 64 scans at 8 cm⁻¹ resolution were collected and averaged. The study involves FT-NIR spectra collected from 32 males and 49 females at the age of 20-65 years (the average age was 37 years for females and 36 years for males). For each psoriasis sample transplanted to a SCID mouse 30 scans at 8 cm⁻¹ resolution were collected and averaged. The study involved spectra from 24 SCID mice transplanted with human psoriasis biopsies. As SCID mice do not have a functional immune system, the human skin biopsies are accepted and may be kept alive for at least 4 weeks. Spectra from the SCID mice were frequently collected prior to and throughout a test period of 4 weeks giving a total of 145 spectra. Throughout this test period 15 of the SCID mice were treated with Pemulen cream placebo and the other 9 SCID mice were treated with Betamethasone ointment.

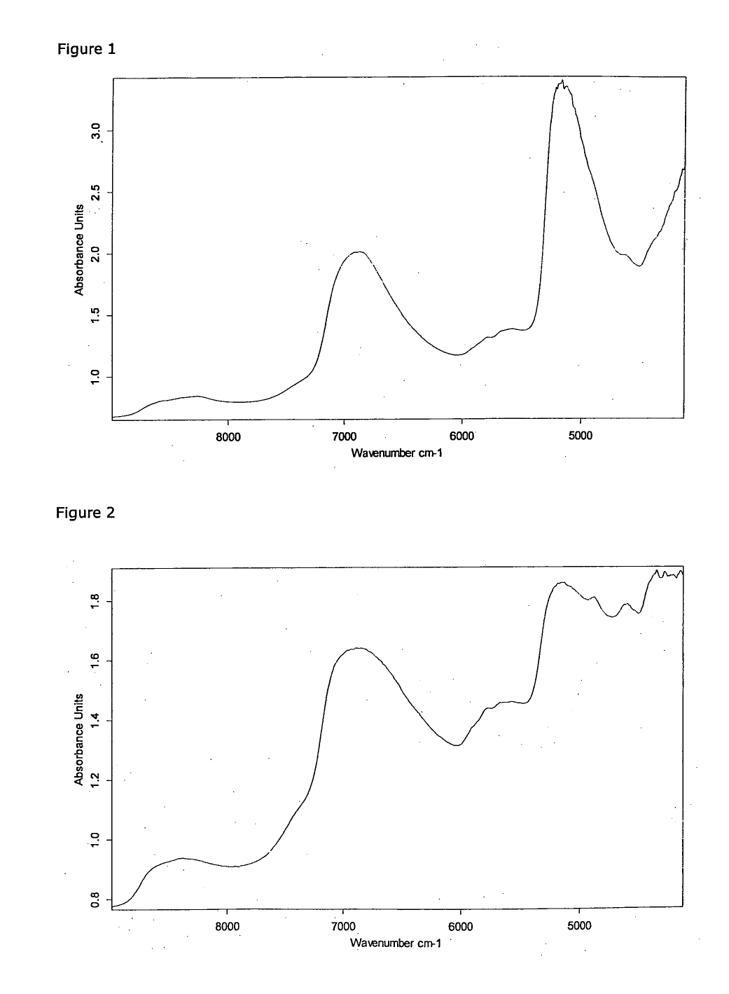

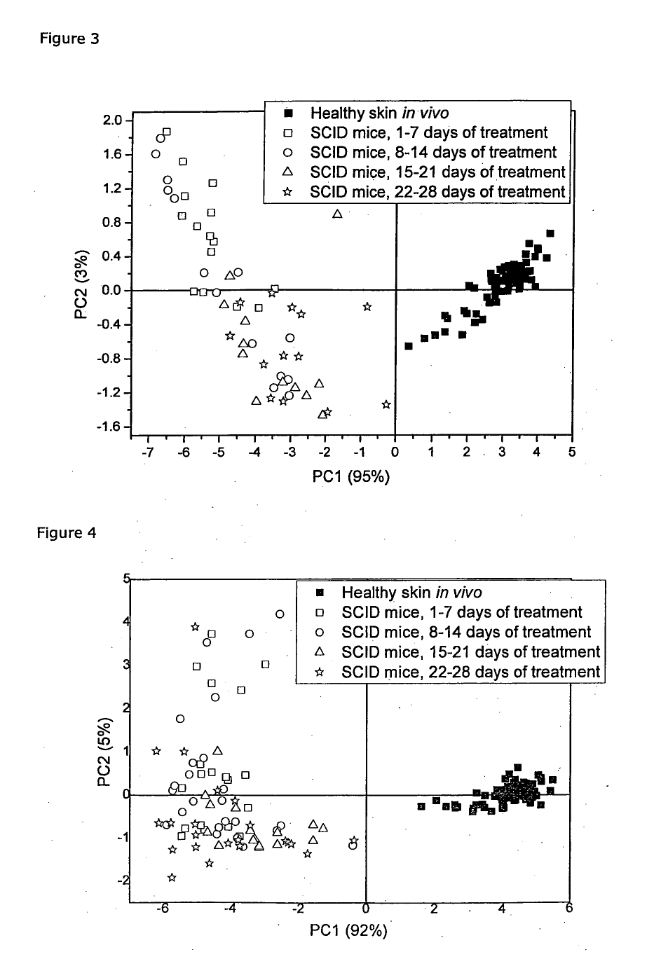

Results: The NIR spectra of healthy skin as shown in FIG. 1 obviously differ from the spectra of untreated psoriasis transplants on SCID mice as shown in FIG. 2. Using a Principal Component Analysis (PCA) on the collected data the scoreplot shows a large separation between the spectra of healthy skin and the spectra of psoriasis transplants cf. FIG. 3, wherein the spectra of healthy skin are shown on the right, whereas the spectra of psoriasis transplants are shown on the left.

Further, for the SCID mice treated with Betamethasone ointment the distance between the scores of the spectra from the SCID mice and the spectra from healthy skin decreases with the number of days the treatment has been performed, indicating a therapeutic effect of the Betamethasone treatment, see FIG. 3.

For the SCID mice treated with Pemulen cream placebo there is no general change in the scores with the number of days the treatment has been performed, cf. FIG. 4. Thus the PCA confirms that the placebo treatment does not in general influence the condition of the skin and thus has no therapeutic effect whereas the betametasone treatment improves the condition of the skin.

### Example 2

A Q-Interline Quant spectrometer equipped with an InGaAs detector (available from Q-Interline) was used to obtain FT-NIR spectra in the region 12500-4000 cm⁻¹. The spectrometer was coupled with a fiber-optic probe which has a circular measurement area of approximately 3 mm in diameter. The measurements were based on diffuse reflectance. For each skin sample 64 scans at 8 cm⁻¹ resolution were collected and averaged. The study involves FT-NIR spectra collected in vivo from 7 psoriasis patients at the age of 29-63 years. For each patient two FT-NIR spectra were recorded at a psoriasis plaque and one FT-NIR spectrum was recorded close by at an asymptomatic skin site with no visual signs of psoriasis. Further, each patient was assigned a local PASI score corresponding to the severity of the measured psoriasis plaque.

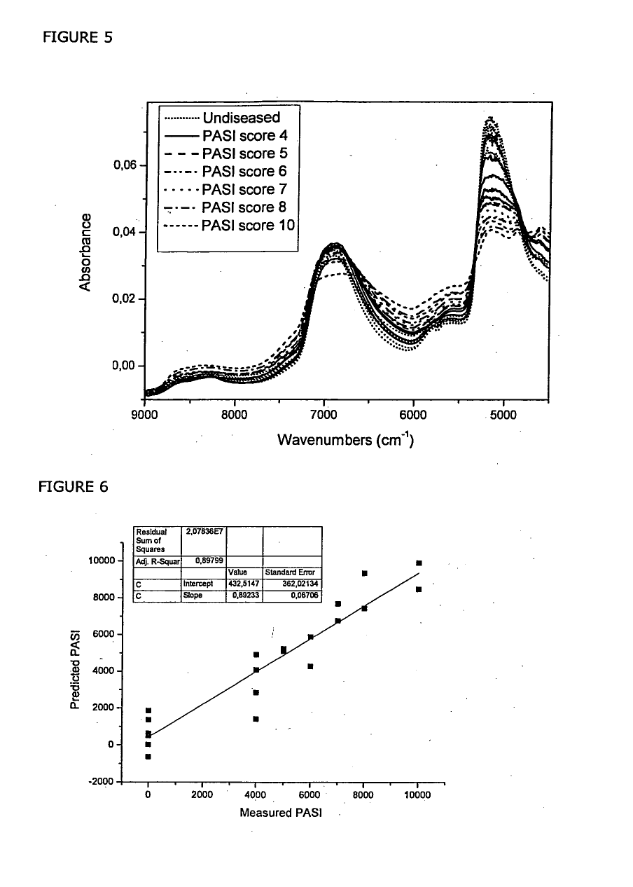

Results: As shown in FIG. 5 the NIR spectra of the asymptomatic skin sites obviously differ from the spectra of the psoriasis plaques. Further, there is a variation in the NIR spectra recorded at the psoriasis skin sites from patients with different local PASI scores. Using Partial Least Squares (PLS) regression it is shown that the variations in the NIR spectra are almost linearly correlated to the local PASI scores assigned to each patients, see FIG. 6.

### CONCLUSION

The results shown in FIGS. 5 and 6 indicate that this method is expected to provide a non-invasive, fast and objective determination of the severity of psoriasis. Since it correlates well with the standard PASI score method of determining the severity of psoriasis and/or the effect of therapy, it may provide a less subjective and time-consuming alternative to recording the PASI score.

1. A method for determining the state of a skin disorder using spectroscopic analysis of the contents and/or structure of one or more of the components water, lipids and proteins in reference skin and in test skin, respectively, said method comprising the steps of:
i) subjecting at least one site of reference skin to Near Infrared (NIR) spectroscopy to determine the contents and/or structure of one or more of the components water, lipids and proteins thereof;
ii) subjecting at least one site of test skin to Near Infrared (NIR) spectroscopy to determine the contents and/or structure of one or more of the components water, lipids and proteins thereof;
iii) recording any deviation in the determinations of the contents and/or structure of one or more of the components water, lipids and proteins at the respective skin sites as a measure of the state of the skin disorder.

2. The method according to claim 1, wherein said reference skin is used as a negative and/or as a positive control.

3. The method according to claim 1 or 2, wherein said reference skin is healthy skin.

4. The method according to claim 1 for determining the state of a skin disorder using spectroscopic analysis of the contents and/or structure of water in reference skin and in test skin, respectively, said method comprising the steps of:
i) subjecting at least one site of reference skin to Near Infrared (NIR) spectroscopy to determine the contents and/or structure of water thereof;
ii) subjecting at least one site of test skin to Near Infrared (NIR) spectroscopy to determine the contents and/or structure of water thereof;
iii) recording any deviation in the determinations of the contents and/or structure of water at the respective skin sites as a measure of the state of the skin disorder.

5. The method according to claim 1 for determining the state of a skin disorder using spectroscopic analysis of the contents and/or structure of lipids in reference skin and in test skin, respectively, said method comprising the steps of:
i) subjecting at least one site of reference skin to Near Infrared (NIR) spectroscopy to determine the contents and/or structure of lipids thereof;
ii) subjecting at least one site of test skin to Near Infrared (NIR) spectroscopy to determine the contents and/or structure of lipids thereof;
iii) recording any deviation in the determinations of the contents and/or structure of lipids at the respective skin sites as a measure of the state of the skin disorder.

6. The method according to claim 1 for determining the state of a skin disorder using spectroscopic analysis of the contents and/or structure of water and lipids in reference skin and in test skin, respectively, said method comprising the steps of:
i) subjecting at least one site of reference skin to Near Infrared (NIR) spectroscopy to determine the contents and/or structure of water and lipids, respectively, thereof;
ii) subjecting at least one site of test skin to Near Infrared (NIR) spectroscopy to determine the contents and/or structure of water and lipids, respectively, thereof;
iii) recording any deviation in the determinations of the contents and/or structure of water and lipids at the respective skin sites as a measure of the state of the skin disorder.

7. The method according to claim 1 for determining the state of psoriasis, contact dermatitis, rosacea, systemic lupus erythematosus, urticaria, atrophied, eczematous or atopic dermatitis skin.

8. The method according to claim 7 for determining the state of psoriasis skin.

9. The method according to claim 1 for determining the effect of treatment of skin disorders, the method comprising repeating steps (i)-(iii) of claim 1, at least once at a later point in time and comparing the determinations recorded at said later point in time with the original determinations to determine any change in the state of the skin disorder being treated.

10. The method according to claim 9 for determining the effect of treatment of psoriasis, contact dermatitis, rosacea, systemic lupus erythematosus, urticaria, atrophied, eczematous or atopic dermatitis skin.

11. The method according to claim 9 or 10 for determining the effect of treatment of psoriasis skin.

12. The method according to claim 1 for determining the state of a skin disorder using spectroscopic analysis of reference skin and test skin further comprising the step of chemometric analysis of the data obtained.

13. The method according to claim 1, wherein the NIR spectroscopy is Fourier Transform Near Infrared (FT-NIR) spectroscopy.

14. A system for determining the state of a skin disorder using spectroscopic analysis of the contents and/or structure of one or more of the components water, lipids and proteins in reference skin and in test skin, respectively, the system comprising:
i) a Near Infrared (NIR) spectroscopy device suitable for subjecting at least one site of reference skin to Near Infrared (NIR) spectroscopy and suitable for subjecting at least one site of test skin to Near Infrared (NIR) spectroscopy to determine the contents and/or structure of one or more of the components water, lipids and proteins in said sites of reference and test skin, respectively;
ii) a recording device for recording any deviation in the determinations of the contents and/or structure of one or more of the components water, lipids and proteins at the respective skin sites;
iii) an assessment device for assessing a determination of the state of the skin disorder on the basis of said deviation; and iv) an output device for outputting said determination of the state of the skin disorder.

15. The system according to claim 14, further comprising a user interface for receiving user input concerning the treatment of the test skin site, the output device further being adapted to monitor the effect of said treatment.

16. An apparatus for determining the state of a skin disorder, the apparatus comprising:
i) an input device for interfacing with a Near Infrared (NIR) spectroscopy device for receiving determinations of the contents and/or structure of one or more of the components water, lipids and proteins in at least one site of reference and test skin, respectively, as obtained by NIR spectroscopy applied to said skin sites by the NIR spectroscopy device;
ii) a recording device for recording any deviation in the determinations of the contents and/or structure of one or more of the components water, lipids and proteins at the respective skin sites;
iii) an assessment device for assessing a determination of the state of the skin disorder on the basis of said deviation; iv) an output device for outputting said determination of the state of the skin disorder.

17. The apparatus according to claim 16, further comprising a user interface for receiving user input concerning the treatment of the test skin site, the output device being further adapted to monitor the effect of said treatment.

18. The method according to claim 4 for determining the effect of treatment of skin disorders, the method comprising repeating steps (i)-(iii) of claim 4 at least once at a later point in time and comparing the determinations recorded at said later point in time with the original determinations to determine any change in the state of the skin disorder being treated.

19. The method according to claim 5 for determining the effect of treatment of skin disorders, the method comprising repeating steps (i)-(iii) of claim 5 at least once at a later point in time and comparing the determinations recorded at said later point in time with the original determinations to determine any change in the state of the skin disorder being treated.

20. The method according to claim 6 for determining the effect of treatment of skin disorders, the method comprising repeating steps (i)-(iii) of claim 6 at least once at a later point in time and comparing the determinations recorded at said later point in time with the original determinations to determine any change in the state of the skin disorder being treated.
